(19) United States
(12) Patent Application Publication
(10) Pub. No.: US 2016/0030179 A1
(43) Pub. Date: Feb. 4, 2016

(54) IMPLANTABLE MEDICAL APPARATUS AND SYSTEMS

(71) Applicant: THE BOARD OF REGENTS OF THE UNIVERSITY OF TEXAS SYSTEM, Austin, TX (US)

(72) Inventors: Ian THOMPSON, San Antonio, TX (US); Yusheng FENG, San Antonio, TX (US); Luis DAVILA, San Antonio, TX (US); Dustin WILSON, San Antonio, TX (US); Michael LASCH, San Antonio, TX (US)

(21) Appl. No.: 14/774,193

(22) PCT Filed: Mar. 14, 2014
(86) PCT No.: PCT/US14/28451

§ 371 Date: Sep. 10, 2015

Related U.S. Application Data
(60) Provisional application No. 61/784,007, filed on Mar. 14, 2013.

(51) Int. Cl. A61F 2/26

(52) U.S. Cl. CPC A61F 2/26

(57) ABSTRACT

An implantable medical apparatus comprising a control unit, a bladder containing a fluid, a ram configured to compress the fluid in the bladder, and a pressure sensor configured to detect a pressure of the fluid in the inflatable bladder. The bladder, ram, pressure sensor and control unit are located in a unitary implant configured for placement within a corporeal body.

### CROSS-REFERENCE TO RELATED APPLICATIONS

This application claims priority to U.S. Provisional Patent Application Ser. No. 61/784,007, filed Mar. 14, 2013, the contents of which are incorporated herein by reference.

### BACKGROUND INFORMATION

As recently as thirty years ago in the urologic surgery arena, the inflatable penile prosthesis may have been the least common form of prosthetic placed for erectile dysfunction. Reasons for the lack of use included the complexity of placement of the prosthesis, which required placement of two inflatable cylinders into the corpora cavernosa, a pump in the scrotum, and a reservoir placed inside the inguinal region, ideally within the abdominal cavity. Placement was often challenging, especially in the patient who had undergone prior abdominal surgery and due to the extensive components and multiple incisions, infections, especially in the diabetic population, were problematic.

In competition with these inflatable penile prostheses (hereafter, IPP) were a range of semi-rigid devices. These devices did not inflate but were flexible and sufficiently rigid to allow for vaginal penetration. They were also easy to implant, requiring only an incision in the corpora. For this reason, the majority of urologists placed them and the number of patients who availed themselves of a prosthetic device was substantial.

For a number of reasons, these semi-rigid prostheses are largely not utilized today. This effectively leaves only the inflatable prostheses as a viable option. One challenge with this situation is that only a very small number of physicians are adept at placing these and troubleshooting problems that can develop. As a result, few patients who could benefit from a prosthetic device actually have one placed. Additionally, the typical IPP currently available has a pump which is difficult to use for patients who are generally elderly with lower strength and have difficulty, often due to obesity or other physical challenges, with inflating the device. For these reasons, few patients who would benefit from a prosthetic device receive one and few physicians who would otherwise be capable to implant one do so and offer the procedure.

Current practice involves three varieties of inflatable devices: self-contained, two-piece and three-piece devices. The American Medical Systems two-piece Ambicor device, the only two-piece in production at present, consists of cylinders that are pre-connected to a ball-shaped pump, which is seated within the scrotum.

Compression of the pump results in transfer of fluid from the back part of the cylinders into the middle portion resulting in rigidity. The deflation mechanism involves bending of the penis in mid-shaft resulting in fluid being returned to the back section. There are currently five different three-piece devices available to address most implant situations. Three-piece inflatable implants have paired cylinders, a small scrotal pump, and a large-volume fluid reservoir (which is placed behind the abdominal wall muscles). American Medical Systems manufactures three devices of this type.

Post-operative complications of penile implant surgery are not uncommon and include but are not limited to implant infection, component failure, device erosion, device migration, sizing problems and auto-inflation.

Urinary incontinence is a problem that affects many patients, children, men, and women. Incontinence of urine is an utterly devastating problem. In the US where access to diapers and pads is generally possible, it is socially unacceptable (often due to the smell), leads to social withdrawal in many patients, and results in tremendous expense. In other countries, patients are exiled from their communities. For patients with severe incontinence, it is best managed with an artificial urinary sphincter (AUS). The current generation of AUS's includes three components: a cuff that is placed around the urethra (or bladder neck in children), a pump that is placed in the scrotum or labia, and a reservoir that is placed in the abdominal cavity. When placed and when working appropriately, it is a transformational event for a patient, allowing them to re-enter society and to resume a normal lifestyle.

Unfortunately, the AUS that is currently available suffers from a number of challenges. The cuff is pressurized at effectively the same pressure for its lifetime: the pressure reservoir in the abdomen is pressurized at the time of the procedure and the only way to change the pressure is to re-operate on the patient. This is also a problem for many patients whose pressure required to maintain continence changes over time. This can happen when the cuff is placed, for example, in the bulbar urethra which, over time, can atrophy, leading to development of new incontinence.

The second problem with the system is that the pump is quite difficult to activate. It is small and requires considerable pressure to be placed in one specific location while the other hand of the patient stabilizes the pump in the scrotum or labia. For many young patients as well as elderly patients, this is a difficult task. In some occasions, as the patient ages, it is necessary to deactivate the cuff to prevent overdistension of the bladder. In the small scrotum or labium, the pump is extremely difficult to activate (and to implant properly).

The final problem, like that of the penile prosthesis, is that the device is difficult to place for many physicians. The most common location for placement is in the bulbar urethra, especially after radical prostatectomy. This requires (generally) an incision in the mid-perineum for placement of the cuff, extension of the incision to create a pocket for the pump in the scrotum, and often requires a second incision in the inguinal region for placement of the pressure reservoir. For many physicians, this is simply too difficult to accomplish, especially if performed only occasionally. For this reason, there is only a small cadre of physicians who currently implant an AUS.

### SUMMARY

Exemplary embodiments of the present disclosure comprise an implantable medical system configured to be surgically implanted in a patient for the treatment of various medical conditions. Specific exemplary embodiments comprise a self-contained, inflatable device configured as a unitary implant which requires simplified placement in patients' bodies. In exemplary embodiments, implant placement can be accomplished in a procedure that would take an average urologist no more than approximately thirty minutes, reducing risk of infection. In exemplary embodiments, the device can be activated externally by a wireless (Bluetooth, radio frequency etc.) control device. The device can also use sensors to measure internal pressure to ensure optimal, individualized inflation.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

Exemplary embodiments of the present disclosure comprise an implantable medical apparatus comprising: an inflatable bladder containing a fluid; a ram configured to compress the fluid; a pressure sensor configured to detect a pressure of the fluid; and a control unit configured to move the ram. In specific embodiments, the inflatable bladder, the ram, the pressure sensor and the control unit are located in an implant configured for placement within a body. In certain embodiments, the control unit comprises a wireless power receiver controller, and in particular embodiments, the control unit comprises an inductor coupled to the wireless power receiver controller. In particular embodiments, the control unit comprises a linear motor configured to move the ram.

In specific embodiments, the control unit comprises a three-phase sine wave generator configured to control a position of the linear motor. In certain embodiments, the control unit comprises a microcontroller unit, which can be powered by a buck circuit in particular embodiments. In particular embodiments, the apparatus may be configured as a penile prosthetic and the inflatable bladder is configured to move from a deflated or flaccid state to an inflated or rigid state when the ram moves toward a distal end of the unitary implant and compresses the fluid. In specific embodiments, the apparatus may be configured as the apparatus may be configured as an artificial unitary sphincter and the inflatable bladder is configured to move from a deflated state to an inflated state when the ram moves toward a distal end of the unitary implant and compresses the fluid.

In certain embodiments, the control circuit is configured to record a target pressure of the fluid when the inflatable bladder is in the inflated or rigid state. In specific embodiments, the control circuit is configured to control the location of the ram to achieve the target pressure of the fluid. The bladder can be configured to move from an inflated or rigid state to a deflated or flaccid state when the ram moves away from a distal end of the implant and decreases the pressure of the fluid in particular embodiments.

In certain embodiments, the pressure sensor is coupled to the ram. Particular embodiments can further comprise a transmission device configured to wirelessly transmit power to the control circuit. In particular embodiments, the transmission device is configured as a belt.

In certain embodiments, the control circuit comprises a linear motor coupled to the ram; the linear motor is configured to move from a first position distal to the distal end to a second position proximal to the distal end; and the control circuit detects the position of the linear motor when the transmission device is initially positioned proximal to the control circuit. In particular embodiments, the control circuit moves the linear motor towards the first position if the linear motor is in the second position when the transmission device is initially positioned proximal to the control circuit. In specific embodiments, the control circuit stops the linear motor when the pressure sensor detects the pressure of the fluid has reached a target value. In particular embodiments, the implantable medical apparatus is configured as a penile prosthetic, and the inflatable bladder moves from a deflated or flaccid state to an inflated or rigid state as the linear motor moves from a first position to a second position. In certain embodiments, the implantable medical apparatus is configured as an artificial unitary sphincter, and the inflatable bladder moves from a deflated state to an inflated state as the linear motor moves from a first position to a second position.

In certain embodiments, the control circuit moves the linear motor towards the second position if the linear motor is in the first position when the transmission device is initially positioned proximal to the control circuit. In particular embodiments, the inflatable bladder moves from an inflated state to a deflated state as the linear motor moves from the second position towards the first position.

Exemplary embodiments of the present disclosure comprise a single piece penile erectile system configured to be surgically implanted in a man for the treatment of erectile impotence. Specific exemplary embodiments comprise a self-contained, inflatable device configured as a unitary implant which requires only placement in the corporeal bodies. In exemplary embodiments, implant placement can be accomplished in a procedure that would take an average urologist no more than approximately thirty minutes, reducing risk of infection. In exemplary embodiments, the device can be activated externally by a wireless (Bluetooth, radio frequency etc.) control device. The device can also use sensors to measure internal pressure to ensure optimal, individualized inflation. In certain embodiments, the corporeal body is a corpus cavernosum.

Exemplary embodiments of the present disclosure comprise an artificial urinary sphincter system configured to be surgically implanted in a patient for the treatment of urinary incontinence. Specific exemplary embodiments comprise a self-contained, inflatable device configured as a unitary implant which requires only placement in one body cavity. In exemplary embodiments, implant placement can be accomplished in a procedure that would take an average urologist no more than approximately thirty minutes, reducing risk of infection. In exemplary embodiments, the device can be activated externally by a wireless (Bluetooth, radio frequency etc.) control device. The device can also use sensors to measure internal pressure to ensure optimal, individualized inflation. In certain embodiments, the body cavity is the abdominal cavity.

The disclosed invention can also be used for the treatment of various other medical conditions by varying the size, shape and placement of the inflatable bladder. Other applications can include but are not limited to:
1. Rectal artificial sphincter for fecal incontinence. Essentially the same system as the AUS with a lager bladder which would use a larger volume of fluid.
2. The present invention could be used to adjust the pressure for the current Lap Band device for bariatric surgery which can be adjusted by the introduction of fluid into the subcutaneous reservoir. There are conditions under which the lap band should be deactivated or when a greater degree of pressure may be required.
3. The present invention could be used as part of a cuff device which could successfully compress and decompress a hollow viscus, that could be used for anti-reflux surgery to replace the Nissen Fundoplication. When a patient is not eating or when the patient becomes supine or asleep, it could be activated. It could be deactivated during the day or when eating.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 shows a partial-section side view according to an exemplary embodiment.

FIG. 2 shows a partial-section perspective view of the embodiment of FIG. 1 in a rigid state.

FIG. 3 shows the embodiment of FIG. 1 implanted in a flaccid and erect state.

FIG. 4 shows a partial-section side view of the embodiment of FIG. 1 in a flaccid state.

FIG. 5 shows a partial-section perspective view of the embodiment of FIG. 1 in a flaccid state.

FIG. 6 shows a partial-section end and side views of the embodiment of FIG. 1 in a rigid state.

FIG. 7 shows a schematic diagram of a control circuit of the embodiment of FIG. 1 and FIG. 8.

FIG. 8 shows a partial-section side view according to an exemplary embodiment.

FIG. 9 shows a line drawing according to an exemplary embodiment.

FIG. 10 shows a partial-section side view illustrating the embodiment of FIG. 8 in an inflated and deflated state.

### DETAILED DESCRIPTION OF ILLUSTRATIVE EMBODIMENTS

### EXAMPLE 1

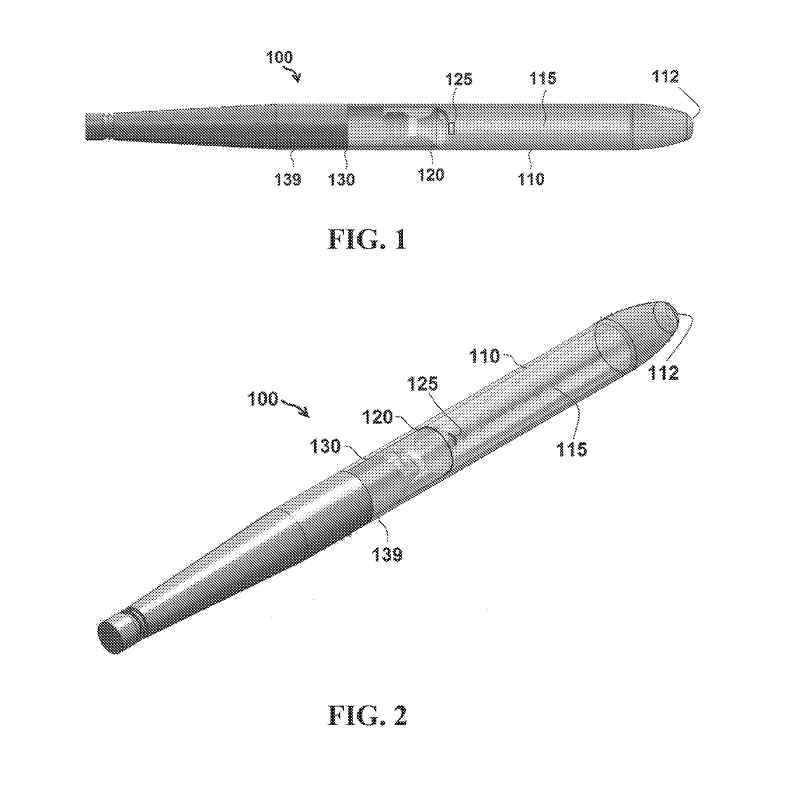

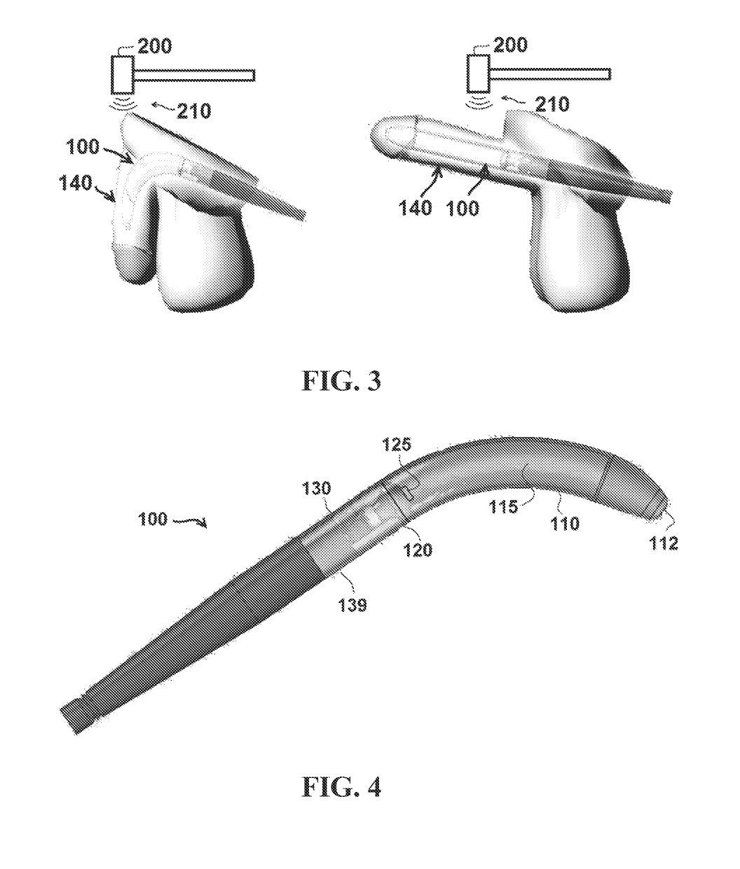

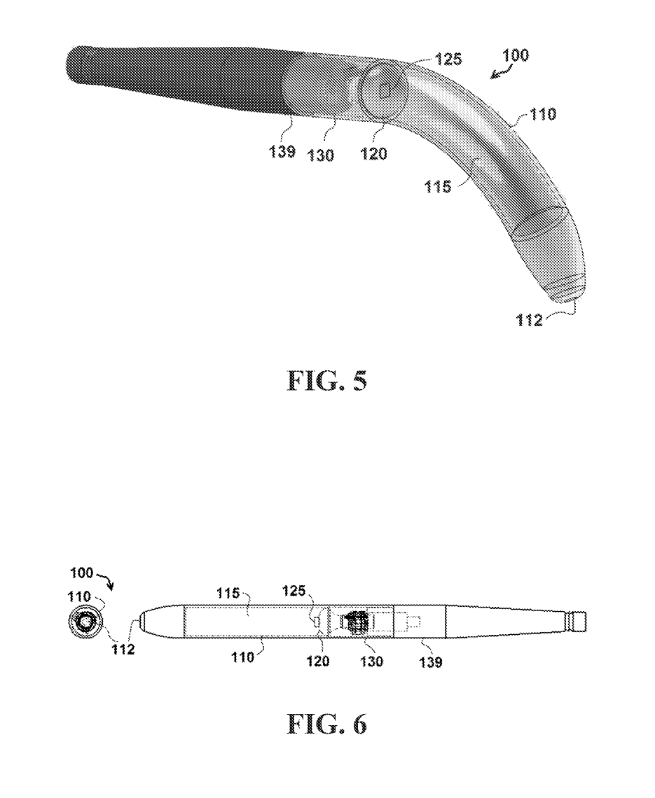

Referring initially to FIGS. 1-6, an exemplary embodiment of a penile prosthetic apparatus is shown configured as an implant 100 comprising an inflatable bladder 110, a ram 120, a pressure sensor 125 and a control unit 130. In this embodiment, control unit 130 is disposed within a generally cylindrical housing 139. In exemplary embodiments, implant 100 is configured as a unitary implant for placement in a corporeal body 140 (e.g. a corpus cavernosum) as shown in FIG. 3, such that no additional equipment located outside of a corporeal body is needed for operation of the apparatus.

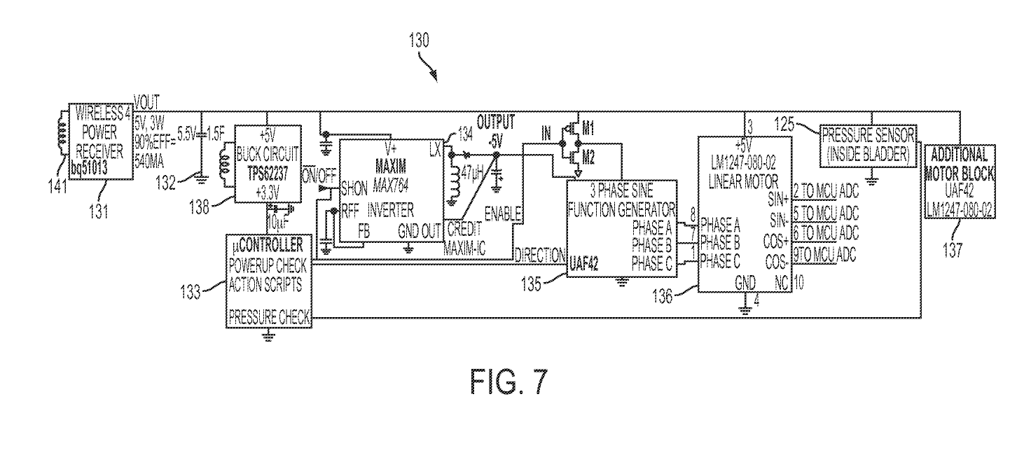

In the embodiment shown, pressure sensor 125 is configured to sense the pressure of a fluid 115 in inflatable bladder 110. In addition, control unit 130 is configured to move ram 120 toward a distal end 112 of implant 112 to increase the pressure of fluid 115. Control unit 130 is also configured to move ram 120 away from distal end 112 to decrease the pressure of fluid 115. In certain embodiments fluid 115 may be a compressible gas such as air or an inert gas such as nitrogen. A schematic diagram of the circuit in control unit 130 is provided in FIG. 7.

During operation, in order to move from a flaccid state to a rigid or erect state (as shown in FIG. 3), a user can place a transmission device 200 onto the skin above the implantation site. In certain embodiments, transmission device 200 can be configured as a belt-type device. In particular embodiments, transmission device 200 is capable of wirelessly transmitting the entire amount of power consumed in the circuit in a wireless signal 210.

Once the user has placed transmission device 200 near implant 100, an internal inductor 141 in control unit 130 can then generate a voltage to be utilized by control unit 130. In a specific embodiment, control unit 130 can comprise a wireless power receiver controller 131 that takes the input voltage and generates a 5V, 3W output at approximately ninety percent efficiency. This can initially charge a capacitor 132 in order to provide control unit 130 with a more consistent power if transmission device 200 is moved during the initial phase.

In exemplary embodiments, after capacitor 132 has been charged there will be sufficient power to enable a microcontroller unit (MCU) 133 in control unit 130. In particular embodiments, MCU 133 is powered by way of a buck circuit 138 in order to maximize the conversion of the 5V to 3.3V. In specific embodiments, the switching supply can have an efficiency of greater than ninety percent.

Upon initial boot up, MCU 133 can check the position sensors of each of a pair of linear motors 136 and 137. Depending on the initial start position of motors 136 and 137, MCU 133 is able to determine the correct direction in which motors 136 and 137 should be moved. In the embodiment shown, an inverter 134 converts the direct current (DC) to alternating current (AC), and three-phase sine wave generator 135 is employed in order to control each motor's position. Three phase sine wave generator 135 outputs three sine waves, each 90 degrees out of phase of each other. MCU 133 is able to output direction information to three-phase sine generator system 135.

Additionally, MCU 133 is able to control the power to the sine wave generator system 135, which can reduce the power required for operation of the apparatus. As control unit 130 is inflating bladder 110, a pressure sensor 125 housed within bladder 110 (e.g. on the front face of ram 120) will monitor pressure of fluid 115 inside bladder 110. If the pressure exceeds a target value, MCU 133 can stop the forward progression of ram 120 and reverse its direction enough to reduce the fluid 115 pressure to be within a desired range of the target pressure. In addition, control circuit 130 can record the target pressure and position ram 120 so that the fluid pressure is maintained at the target pressure.

In order to achieve the downward, deflation motion of bladder 110, the user can again place wireless transmission device 200 above implant 100. Once MCU 133 turns on, it will do the same initial position check as before, and it will realize that it should reverse the direction of motors 136 and 137. MCU 133 can then enable and send the appropriate direction information to sine wave generator 135 which will reverse the direction to the rest position. Pressure sensor 125 can be ignored during the deflation operation, as it is not needed during the pressure reduction stage.

In a specific embodiment, wireless power receiver controller 131 may be a model BQ51013 model available from Texas Instruments and motors 136 and 137 may be a Faulhaber QuickShaft LM 1247-080-02. In addition, buck circuit 138 may be a Texas Instruments TPS62237 model, while sine wave generator system 135 is a Texas Instruments UAF42 model and MCU 131 may be a Texas Instruments Safety ARM Processor.

Apparatus and methods according to exemplary embodiments of the present disclosure provide for numerous benefits over existing devices and methods. For example, exemplary embodiments can be implanted by an urologist without the need for specialized or complicated equipment, and no incisions or invasion of the abdominal cavity are required. The time and cost of implantation can therefore be reduced, and the procedure performed on an outpatient basis, reducing the need for patients to require an overnight stay for observation.

In addition, if a cylinder malfunctions, it is a simple approach to replace, without the need for re-pressurization of the entire liquid flow system as is required in the current generation IPPs. Furthermore, if an infection of one component occurs, that single component can be removed and then replaced rather than the need to remove/replace the entire IPP.

Exemplary embodiments also allow an elderly user to inflate the device without concern for strength, obesity, or other causes that currently prevent inflation of the difficult-to-activate typical IPP pump. Furthermore, the system pressurizes based on internal sensors to an appropriate target pressure, which can reduce the likelihood of over-inflation and allow for programming of the system to achieve the similar result each time. In addition, with some patients, the scrotum is too small to properly accommodate the pump with the current IPP. This is not an issue with exemplary embodiments of this disclosure as, the implant is a unitary device configured for placement within the corporeal bodies.

Additional benefits and advantages of exemplary embodiments include the configuration as a single device that allows for easier surgical insertion and decrease in postoperative complications. In addition, a shorter healing period is also achievable with the unitary implant configuration. Exemplary embodiments further provide for greater control over the rigidity of erect state and a faster time to achieve the erect state. In addition, the automated process associated with exemplary embodiments provides for easier use.

### EXAMPLE 2

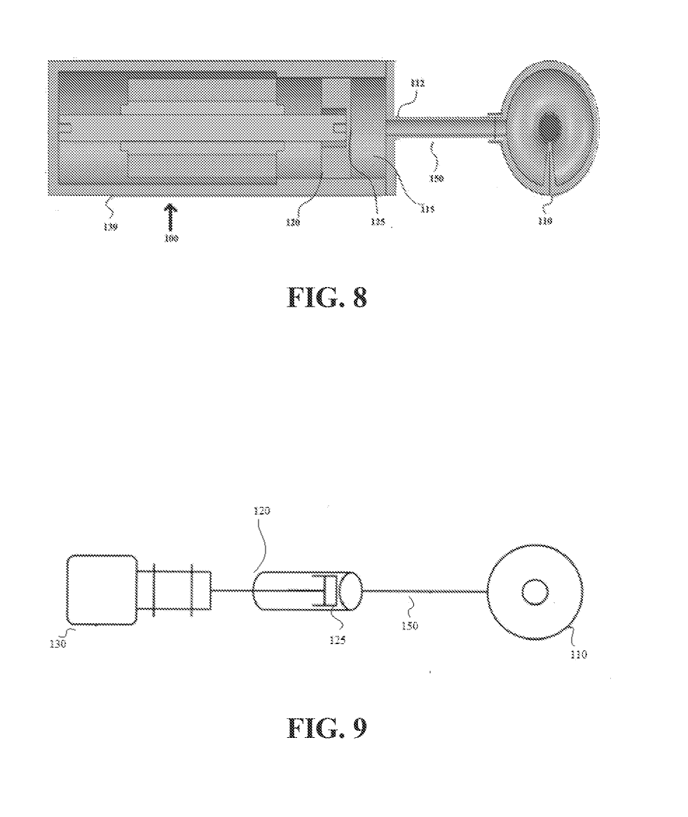

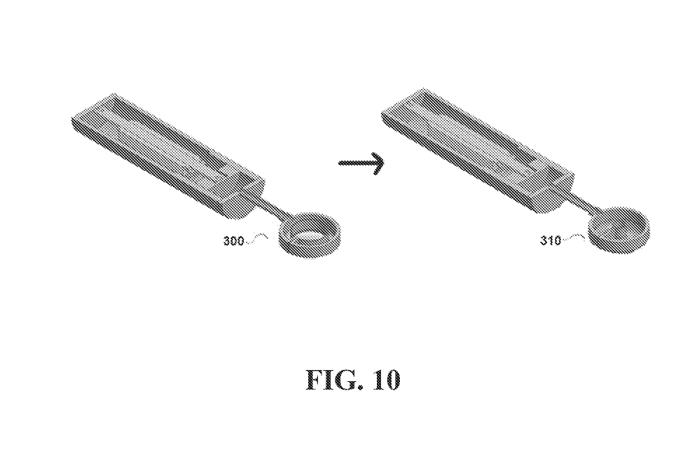

Referring initially to FIGS. 8-9, an exemplary embodiment of an artificial urinary sphincter apparatus is shown configured as an implant 100 comprising an inflatable bladder 110, a ram 120, a pressure sensor 125 and a control unit 130. In this embodiment, control unit 130 is disposed within a generally cylindrical housing 139, and inflatable bladder 110 is coupled to the distal end of rest of the implant by fluid line 150.

In the embodiment shown in FIGS. 8-9, pressure sensor 125 is configured to sense the pressure of a fluid 115 in inflatable bladder 110. In addition, control unit 130 is configured to move ram 120 toward a distal end 112 of implant 112 to increase the pressure of fluid 115. Control unit 130 is also configured to move ram 120 away from distal end 112 to decrease the pressure of fluid 115. In certain embodiments fluid 115 may be a compressible gas such as air or an inert gas such as nitrogen. A schematic diagram of the circuit in control unit 130 is provided in FIG. 7.

During operation, in order to move from a deflated state 300 to an inflated state 310 (as shown in FIG. 9), a user can place a transmission device onto the skin above the implantation site. In certain embodiments, transmission device can be configured as a belt-type device. In particular embodiments, transmission device is capable of wirelessly transmitting the entire amount of power consumed in the circuit in a wireless signal.

Once the user has placed transmission device near implant 100, an internal inductor 141 in control unit 130 can then generate a voltage to be utilized by control unit 130. In a specific embodiment, control unit 130 can comprise a wireless power receiver controller 131 that takes the input voltage and generates a 5V, 3W output at approximately ninety percent efficiency. This can initially charge a capacitor 132 in order to provide control unit 130 with a more consistent power if transmission device 200 is moved during the initial phase.

In exemplary embodiments, after capacitor 132 has been charged there will be sufficient power to enable a microcontroller unit (MCU) 133 in control unit 130. In particular embodiments, MCU 133 is powered by way of a buck circuit 138 in order to maximize the conversion of the 5V to 3.3V. In specific embodiments, the switching supply can have an efficiency of greater than ninety percent.

Upon initial boot up, MCU 133 can check the position sensors of each of a pair of linear motors 136 and 137. Depending on the initial start position of motors 136 and 137, MCU 133 is able to determine the correct direction in which motors 136 and 137 should be moved. In the embodiment shown, an inverter 134 converts the direct current (DC) to alternating current (AC), and three-phase sine wave generator 135 is employed in order to control each motor's position. Three phase sine wave generator 135 outputs three sine waves, each 90 degrees out of phase of each other. MCU 133 is able to output direction information to three-phase sine generator system 135.

Additionally, MCU 133 is able to control the power to the sine wave generator system 135, which can reduce the power required for operation of the apparatus. As control unit 130 is inflating bladder 110, a pressure sensor 125 housed within bladder 110 (e.g. on the front face of ram 120) will monitor pressure of fluid 115 inside bladder 110. If the pressure exceeds a target value, MCU 133 can stop the forward progression of ram 120 and reverse its direction enough to reduce the fluid 115 pressure to be within a desired range of the target pressure. In addition, control circuit 130 can record the target pressure and position ram 120 so that the fluid pressure is maintained at the target pressure.

In order to achieve the, deflation motion of bladder 110, the user can again place wireless transmission device above implant 100. Once MCU 133 turns on, it will do the same initial position check as before, and it will realize that it should reverse the direction of motors 136 and 137. MCU 133 can then enable and send the appropriate direction information to sine wave generator 135 which will reverse the direction to the rest position. Pressure sensor 125 can be ignored during the deflation operation, as it is not needed during the pressure reduction stage.

In a specific embodiment, wireless power receiver controller 131 may be a model BQ51013 model available from Texas Instruments and motors 136 and 137 may be a Faulhaber QuickShaft LM 0830 Series. In addition, buck circuit 138 may be a Texas Instruments TPS62237 model, while sine wave generator system 135 is a Texas Instruments UAF42 model and MCU 131 may be a Texas Instruments Safety ARM Processor. In a specific embodiment the inflatable bladder 110 and fluid line 150 may be the same as found in the AMS 800™ Urinary Control System.

After implantation the patient could inflate the cuff to a point so that no incontinence was noted. For example, during the evening when most patients have no incontinence, the device could be configured to have an ‘evening’ mode could be used that would reduce urethral pressure. On the other hand, when playing tennis, the device could be configured to have an ‘active’ mode could be used to increase the pressure.

Exemplary embodiments also allow an elderly user to activate the device without concern for strength, obesity, or other causes that currently prevent activation of the difficult-to-activate typical AUS pump. Furthermore, the system pressurizes based on internal sensors to an appropriate target pressure, which can reduce the likelihood of over or under-inflation and allow for programming of the system to achieve the similar result each time. In addition, with some patients, the scrotum is too small to properly accommodate the pump with the current AUS. This is not an issue with exemplary embodiments of this disclosure as, the implant is a device configured for placement without the need of a separate fluid reservoir or manual pump.

All of the apparatus, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
U.S. Patent Publication 2003/0125768
U.S. Patent Publication 2012/0022323
U.S. Pat. No. 3,954,102
U.S. Pat. No. 4,009,711
U.S. Pat. No. 4,201,202
U.S. Pat. No. 4,318,396
U.S. Pat. No. 4,342,308
U.S. Pat. No. 4,407,278
U.S. Pat. No. 5,062,416
U.S. Pat. No. 6,475,137
U.S. Pat. No. 7,011,624
U.S. Pat. No. 7,367,938
U.S. Pat. No. 7,338,437
U.S. Pat. No. 7,407,481
U.S. Pat. No. 7,442,165
U.S. Pat. No. 8,126,558
U.S. Pat. No. 8,290,594
U.S. Pat. No. 8,287,444

1. An implantable medical apparatus comprising:
an inflatable bladder containing a fluid;
a ram configured to compress the fluid;
a pressure sensor configured to detect a pressure of the fluid; and
a control unit configured to move the ram, wherein the inflatable bladder, the ram, the pressure sensor and the control unit are located in a unitary implant configured for placement within a corporeal body.

2. The implantable medical apparatus of claim 1 wherein the control unit comprises a wireless power receiver controller.

3. The implantable medical of claim 2 wherein the control unit comprises an inductor coupled to the wireless power receiver controller.

4. The implantable medical apparatus of claim 2 wherein the control unit comprises a linear motor configured to move the ram.

5. The implantable medical apparatus of claim 4 wherein the control unit comprises a three-phase sine wave generator configured to control a position of the linear motor.

6. The implantable medical apparatus of claim 2 wherein the control unit comprises a microcontroller unit.

7. The implantable medical apparatus of claim 6 wherein the microcontroller unit is powered by a buck circuit.

8. The implantable medical apparatus of claim 1 wherein the implantable medical apparatus is configured as a penile prosthetic, and wherein the inflatable bladder is configured to move from a flaccid state to a rigid state when the ram moves toward a distal end of the unitary implant and compresses the fluid.

9. The implantable medical apparatus of claim 1 wherein the implantable medical apparatus is configured as an artificial urinary sphincter, and wherein the inflatable bladder is configured to move from a deflated state to an inflated state when the ram moves toward a distal end of the unitary implant and compresses the fluid.

10. The implantable medical apparatus of claim 8 wherein the control circuit is configured to record a target pressure of the fluid when the inflatable bladder is in the rigid state.

11. The implantable medical apparatus of claim 10 wherein the control circuit is configured to control the location of the ram to achieve the target pressure of the fluid.

12. The implantable medical apparatus of claim 1 wherein the bladder is configured to move from an inflated state to a deflated state when the ram moves away from a distal end of the unitary implant and decreases the pressure of the fluid.

13. The implantable medical apparatus of claim 1 wherein the pressure sensor is coupled to the ram.

14. The implantable medical apparatus of claim 1 further comprising a transmission device configured to wirelessly transmit power to the control circuit.

15. The implantable medical apparatus of claim 14 wherein transmission device is configured as a belt.

16. The implantable medical apparatus of claim 14 wherein:
the control circuit comprises a linear motor coupled to the ram;
the linear motor is configured to move from a first position distal to the distal end to a second position proximal to the distal end; and
the control circuit detects the position of the linear motor when the transmission device is initially positioned proximal to the control circuit.

17. The implantable medical apparatus of claim 16 wherein the control circuit moves the linear motor towards the first position if the linear motor is in the second position when the transmission device is initially positioned proximal to the control circuit.

18. The implantable medical apparatus of claim 17 wherein the control circuit stops the linear motor when the pressure sensor detects the pressure of the fluid has reached a target value.

19. The implantable medical apparatus of claim 17 wherein the implantable medical apparatus is configured as a penile prosthetic, and wherein the inflatable bladder moves from a flaccid state to a rigid state as the linear motor moves from a first position to a second position.

20. The implantable medical apparatus of claim 17 wherein the implantable medical apparatus is configured as an artificial urinary sphincter, and wherein the inflatable bladder moves from a deflated state to an inflated state as the linear motor moves from a first position to a second position.

21. The implantable medical apparatus of claim 16 wherein the control circuit moves the linear motor towards the second position if the linear motor is in the first position when the transmission device is initially positioned proximal to the control circuit.

22. The implantable medical apparatus of claim 21 wherein the inflatable bladder moves from a rigid state to a flaccid state as the linear motor moves from the second position towards the first position.

23. The implantable medical apparatus of claim 1 wherein the corporeal body is a corpus cavernosum.
